(19) United States
(12) Patent Application Publication
(10) Pub. No.: US 2022/0236267 A1
(43) Pub. Date: Jul. 28, 2022

(54) FILTER TOXIN AND ANTIGEN DETECTOR

(71) Applicants: Brian Kamradt, Indianapolis, IN (US); Chris Zeller, Springboro, OH (US); Charles Zeller, Fort Lauderdale, FL (US)

(72) Inventors: Brian Kamradt, Indianapolis, IN (US); Chris Zeller, Springboro, OH (US); Charles Zeller, Fort Lauderdale, FL (US)

(21) Appl. No.: 17/615,026

(22) PCT Filed: May 28, 2020
(86) PCT No.: PCT/US2020/035006

§ 371 Date: Nov. 29, 2021

Related U.S. Application Data
(60) Provisional application No. 62/865,923, filed on Jun. 24, 2019.
(60) Provisional application No. 62/853,703, filed on May 28, 2019.

(51) Int. Cl. G01N 33/543; G01N 1/22; G01N 15/06

(52) U.S. Cl. CPC G01N 33/54388; G01N 2001/2223; G01N 15/0618; G01N 1/2205

(57) ABSTRACT

A filter toxin and antigen detector assembly for detecting the presence or absence of toxins or antigens within air handling systems, ventilators, respirators, continuous positive airway pressure devices (CPAP), and bilevel positive airway pressure (BIPAP) devices is disclosed. The filter toxin and antigen detectors may be attached to an air filter, or placed (such as in the form of a test cartridge or test strip) onto a port or other portion of a respiration device to determine if the device is clean for further medical use. The filter toxin and antigen detectors disclosed herein utilize lateral flow immunochromatographic assay technology having a sample window allowing air flow therethough. The filter toxin and antigen detector will give an immediate presence indication, such as by changing color, thus providing a fast indication of whether or not harmful toxins or antigens are present within an airborne environment.

### PRIORITY

This PCT patent application is related to, and claims the priority benefit of, a) U.S. Provisional Patent Application No. 62/865,923, filed on Jun. 24, 2019, and b) U.S. Provisional Patent Application No. 62/853,703, filed on May 28, 2019. The contents of each of these applications are incorporated herein directly and by reference.

### BACKGROUND

Air quality and the detection of the presence of harmful antigens (such as mold and other inhaled toxins) is a rising health concern. The ability to detect the presence of harmful antigens or toxins in our air is becoming increasingly important in almost every industry, including in residential, commercial, medical, institutional, and military environments, and represents a growing public health need.

While some antigen and toxin detection kits currently exist, they have several disadvantages, including being expensive, requiring extensive testing and/or analysis time (often needing to be sent to a lab for later analysis), are specific for detection of only one certain type of antigen or toxin, are only used when a specific health concern arises (i.e., not on a continuous monitoring basis), or actually contain a medium desirable to antigens and toxins to feed on and to grow. It would thus be desirable to have an antigen and toxin detection kit which is inexpensive and capable of providing an immediate result (without having to be sent to a lab for later analysis), while providing detection for a variety of different types of antigens or toxins, without actually encouraging the growth of harmful antigens or toxins.

It would further be desirable to have the ability to identify harmful toxins or antigens circulating within an airborne environment by using existing air filtration systems (and the air filters therein) coupled with the addition of a disposable, and affordable detectors. The filter toxin and antigen detectors disclosed herein may be readily coupled to any existing air filtration/handling/purification/HVAC system, as well as onto any ventilator or respiration devices, such as onto continuous positive airway pressure (CPAP) and bilevel positive airway pressure devices (BIPAP), thus preventing redesign or retrofitting of expensive existing systems.

The filter toxin and antigen detectors disclosed herein have a uniform/universal design to allow a consumer to detect the presence of harmful toxins or antigens on a continuous basis during routine air filter exchanges (of an air filtration/HVAC system, for example). During a routine air filter replacement, a consumer would simply depress an attached saline ampule (overlying the toxin filter cartridge) in order to start or initiate the filter toxin and antigen detector (i.e., lateral flow immunochromatographic assay). In this manner, the air quality can be monitored continuously, or during regular intervals of each routine air filter replacement. Automated versions of this filter toxin and antigen detector could be further employed to provide the ability for regularly scheduled air quality sampling within industrial, military, medical, or commercial settings. Additionally, filter toxin and antigen detectors (i.e., the lateral flow immunoassays therein) may further be customized to detect antibodies and/or DNA to the desired antigen or toxin.

The filter toxin and antigen detectors disclosed herein also provide an immediate result to indicate if a particular harmful toxin or antigen is present, such as by displaying a color change. The detectors themselves may be removably attached to, or integrated into, existing air filters, such as by adhesives and/or hook and loop fasteners, for example. In some embodiments, the detectors may be coupled with a fan to pull the air into (and through) the lateral assay portion of the detector. In these embodiments, the detector may further be coupled with a photo sensor which can recognize color change and then send an alarm or alert to a smart phone using a corresponding application (i.e., App). Additionally, the detectors disclosed herein may further be configured to detect viruses, bacteria, and/or other pathogens, such as those impacting patient health in hospitals, resident health in retirement centers, student health in schools, etc. These detectors may also be configured to detect other potentially harmful airborne elements, such as asbestos, lead-based paint (released into the air during sanding, etc.), radon (or other radiation), pollen, pat danger, etc.

In additional embodiments, the filter toxin and antigen detectors disclosed herein may be used on positive pressure airway devices, including respiration/ventilation/breathing devices, including continuous positive airway pressure (CPAP) devices, and bilevel positive airway pressure devices (BIPAP). In these embodiments, the filter toxin and antigen detectors may be integrated into a cassette, test cartridge, or test strip that fits onto a port (such as the inlet/inhalation port or expiratory end) of a respiration/ventilation/breathing/CPAP/BIPAP device to detect/test if the machine is clean of harmful toxins, antigens, viruses, bacteria, etc. The detectors may provide a quick test result (within 10-15 minutes) of device cleanliness or contamination. These detectors can reduce the rate of ventilator associated pneumonia (VAP) in medical facilities, could also easily be used on home CPAP/BIPAP machines, and could quickly become the standard of care in the monitoring of respiratory equipment cleanliness and safety.

The ability to monitor air quality for the presence of harmful toxins or antigens may be utilized for a multitude of scenarios, including, but not limited to residential or commercial detection of toxic mold, hospital or heath care monitoring for infectious airborne diseases (such as Tuberculosis, Norovirus, or Legionnaire's disease), identification of biologic toxin exposure (such as Anthrax, Ebola, or Smallpox virus in military installations), identification of airborne communicable diseases in areas of public transit (such as airliners or cruise ships), identification of bacterial pathogens causing ventilator associated pneumonia (the most common nosocomial infection of critically ill patients) etc. within respirators, ventilators, CPAP/BIPAP devices, etc.

Therefore, it would certainly be desirable to have a cost-effective means for monitoring air quality and cleanliness through existing air handling systems, and in respiration/ventilation/breathing/CPAP/BIPAP devices, on a continuous basis, using a filter toxin and antigen detector as disclosed herein.

### BRIEF SUMMARY OF THE INVENTION

In a first embodiment, a filter toxin and antigen detector for detecting the presence or absence of target airborne particles, comprises: at least one lateral flow immunochromatographic assay having a filter sample window therein to provide a path for airflow therethrough, wherein the filter sample window collects airborne particles flowing therethrough for use in the at least one lateral flow immunochromatographic assay; and wherein the at least one lateral flow immunochromatographic assay indicates the presence or absence of the target airborne particles.

In another embodiment, a filter toxin and antigen detector for detecting the presence or absence of target airborne particles, comprises: at least one lateral flow immunochromatographic assay based on a series of capillary beds for transporting fluid spontaneously, comprising: a filter sample window therein to collect airborne particles within an airflow pathway of an airborne environment; a fluid configured to activate the at least one lateral flow immunochromatographic assay; a conjugate pad having antibodies specific to the target airborne particles, such that a chemical reaction is initiated between the antibodies in the conjugate pad and the fluid containing the target airborne particles; and a test result stripe, wherein the chemical reaction and fluids passing through the test result stripe cause a color change in the test result stripe, wherein the color change is indicative of the presence or absence of the target airborne particles.

In another embodiment, a method of using a filter toxin and antigen detector to test for target particulates within an airborne environment, comprises: positioning the detector relative to a substrate, wherein the detector comprises at least one filter sample window configured to collect particulates from the airborne environment, and wherein the filter sample window is within an airflow path of the airborne environment; activating the detector by exposing it to a liquid, wherein the liquid contacts the filter sample window and a chemical reaction begins; and reading a visual indication on the detector to determine if the specific particulates are present or absent within the airborne environment.

An exemplary filter toxin and antigen detector for detecting the presence or absence of target airborne particles of the present disclosure comprises at least one lateral flow immunochromatographic assay having a filter sample window therein, the filter sample window configured to collect target airborne particles flowing through an airborne environment for use in the at least one lateral flow immunochromatographic assay, and wherein the at least one lateral flow immunochromatographic assay indicates the presence or absence of the target airborne particles. In an exemplary embodiment, the detector further comprises adhesive thereon for attachment to an air filter. In an exemplary embodiment, the detector further comprises a hook and loop fastening system thereon for attachment to an air filter. In an exemplary embodiment, the target airborne particles comprise at least one of antibodies, antigens, toxins, biomarkers, pathogens, viruses, bacteria, asbestos, lead, radiation, radon, pollen, pet dander, or allergens. In an exemplary embodiment, the filter sample window is disposed at an angle at or approximately 90 degrees relative to an airflow pathway through the airborne environment. In an exemplary embodiment, the presence or absence of the target airborne particles is indicated by a color change. In an exemplary embodiment, the at least one lateral flow immunochromatographic assay is based on a series of capillary beds for transporting fluid spontaneously. In an exemplary embodiment, the at least one lateral flow immunochromatographic assay further comprises a fluid configured to activate the at least one lateral flow immunochromatographic assay, a conjugate pad having antibodies specific to the airborne particles, such that a chemical reaction is initiated between the antibodies in the conjugate pad and the fluid containing the airborne particles, and a test result stripe, wherein the chemical reaction and the fluid passing through the test result stripe cause a color change in the test result stripe, wherein the color change is indicative of the presence or absence of the target airborne particles. In an exemplary embodiment, the fluid is a liquid sample releasably stored within a glass ampule, wherein the liquid sample is deployed by crushing the glass ampule so that it flows spontaneously. In an exemplary embodiment, the liquid sample comprises saline.

In an exemplary embodiment, the fluid comprises moisture or humidity already present within an airborne environment. In an exemplary embodiment, the conjugate pad comprises dried bio-active particles in a salt-sugar matrix. In an exemplary embodiment, the test result stripe has a thickness which may be quantified to determine concentration of the target airborne particles. In an exemplary embodiment, the detector further comprises a control stripe adjacent the test result stripe, the control figure used to ensure the detector is operating properly.

In an exemplary embodiment, the detector further comprises a thin plastic overlay to protect the integrity of the lateral flow immunochromatographic assay. In an exemplary embodiment, the detector further comprises an adhesive backing for attaching the detector to another surface. In an exemplary embodiment, the detector further comprises a hook and loop fastener for attaching the detector to another surface. In an exemplary embodiment, the lateral flow immunochromatographic assay is designed to be arcuate, similar to an inlet port of a ventilation machine, and wherein the test result stripe is also arcuate. In an exemplary embodiment, the at least one lateral flow immunochromatographic assay comprises three separate lateral flow immunochromatographic assays. In an exemplary embodiment, the at least one lateral flow immunochromatographic assay comprises multiple lateral flow immunochromatographic assays, and wherein each lateral flow immunochromatographic assay of the multiple lateral flow immunochromatographic assays tests for different target airborne particles, thus providing multiple unique tests on the detector. In an exemplary embodiment, the at least one lateral flow immunochromatographic assay comprises multiple lateral flow immunochromatographic assays, and wherein each lateral flow immunochromatographic assay of the multiple lateral flow immunochromatographic assays tests for the same target airborne particles, thus providing multiple redundant tests on the detector.

In an exemplary embodiment of a method of using the filter toxin and antigen detector of the present disclosure to test for the target particulates within an airborne environment, the method comprises the steps of positioning the detector relative to a substrate and so that the filter sample window is within an airflow path of the airborne environment, activating the detector by exposing it to a fluid, wherein the fluid contacts the filter sample window to initiate a chemical reaction, and reading a visual indication on the detector to determine if the target particulates are present or absent within the airborne environment. In an exemplary method, method of claim 22, wherein the step of activating the detector is further performed by facilitating the release of the fluid from within a glass ampule, by crushing the glass ampule to release the fluid. In an exemplary method, the step of activating the detector further comprises exposing the detector to moisture or humidity. In an exemplary method, wherein the step of activating the detector further comprises attaching the detector to a ventilation device such that humidity or moisture already present within the ventilation device serves as the fluid.

In an exemplary method, the step of positioning the detector relative to the substrate further comprises coupling an adhesive on the detector with the substrate. In an exemplary method, the step of positioning the detector relative to the substrate further comprises activating an adhesive on the detector so that the detector securely couples to a substrate. In an exemplary method, the step of reading a visual indication comprises reading a color change on a test stripe area of the at least one lateral flow immunochromatographic assay. In an exemplary method, the step of reading a visual indication further comprises comparing a test color change line to a control color change line on a stripe area of the at least one lateral flow immunochromatographic assay. In an exemplary method, the step of positioning the detector comprises positioning the filter sample window so that it is at or about a 90 degree angle relative to an airflow path through the airborne environment. In an exemplary method, the chemical reaction begins when the fluid contacts the filter sample window and a conjugate within a conjugate pad of the at least one lateral flow immunochromatographic assay. In an exemplary method, the chemical reaction is initiated when the fluid contacts the filter sample window and a conjugate within a conjugate pad of the at least one lateral flow immunochromatographic assay, wherein the conjugate pad contains antibodies specific to the target particulates within the airborne environment.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 illustrates a perspective view of an air filter having an attached exemplary filter toxin and antigen detector;

FIG. 2 illustrates a perspective view of a ventilator having an attached exemplary filter toxin and antigen detector;

FIG. 3 illustrates a perspective view of a portion of a CPAP having an attached exemplary filter toxin and antigen detector,

FIG. 4 illustrates a perspective view of an exemplary filter toxin and antigen detector;

FIG. 5 illustrates a perspective view of an exemplary filter toxin and antigen detector;

FIG. 6 illustrates an enlarged perspective view of the lateral flow assay portion of an exemplary filter toxin and antigen detector,

FIG. 7 illustrates a side view of an exemplary filter toxin and antigen detector;

FIG. 8 illustrates an exploded perspective view of an air filter having an attached exemplary filter toxin and antigen detector, and

FIG. 9 illustrates an enlarged, exploded, perspective view of an air filter having an attached exemplary filter toxin and antigen detector.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes various filter toxin and antigen detectors (also in the form of cassettes, test cartridges, or test strips) for detecting the presence of harmful airborne toxins or antigens, as well as systems, and methods for detecting the presence or absence of toxins or antigens trapped within a filter of an air handling system or within respiration/ventilation/breathing/CPAP/BIPAP devices or within any other device or system having an air flow.

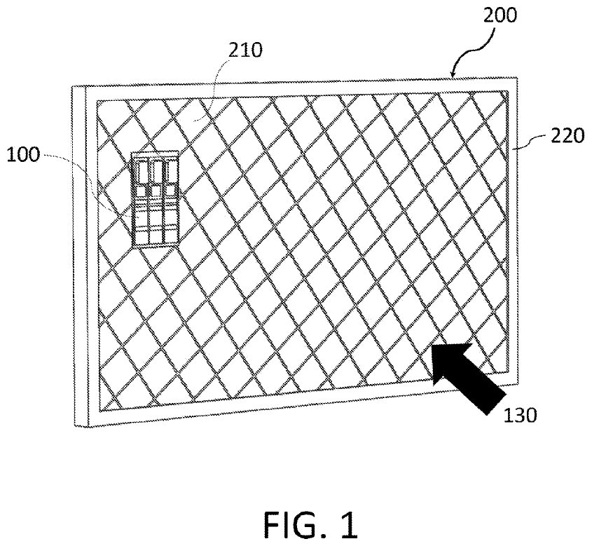

In a first embodiment, shown in FIG. 1, an exemplary filter toxin and antigen detector 100 (or test cartridge 100) is shown disposed on (or attached to) an air filter 200, for detecting the harmful presence of toxins and antigens within an airborne environment. Generally, the filter toxin and antigen detector 100 may be placed on an air filter 200 within an air handling system, such as a furnace, air conditioner, humidifier, a room air purifier, or on an inhalation, expiratory end, or filter of a respiration/ventilation/breathing/CPAP/BIPAP device. However, it should also be understood that the filter toxin and antigen detectors 100 disclosed herein may also be used and applied in other comparable systems, or any systems having air flow therethrough, including any devices where periodic changing of a filter is necessary.

Generally, the filter 200 of an air handling system includes a filter media 210 surrounded and contained within a filter frame 220, as shown in FIG. 1. The filter frame 220 generally surrounds the filter media 210 and may be constructed of paper, chipboard, cardboard, paperboard, boxboard, film, metal or plastic. The entire air filter 200 may be disposable, or only the filter media 210 may be disposable, such that the filter frame 220, or the entire filter, may be reusable.

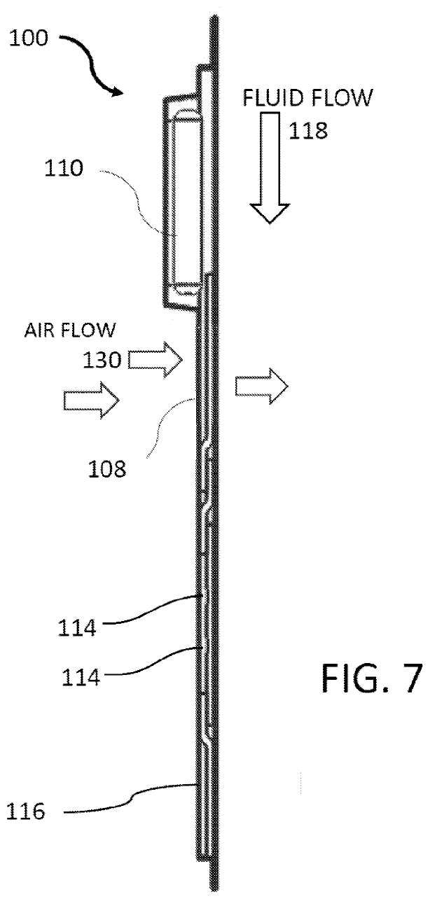

In operation, an air filter 200 is inserted into a housing of an air handling system, usually within a slot designed to closely engage the filter frame 220 of the filter 200. Depending on the particular air handling system, the housing may be a portion of a furnace, an air conditioner, a humidifier, or a room air purifier. The housing itself also contains a fan, for pulling air in through the air filter 200, such that the air will pass through the filter media 210 of the air filter 200. As the air passes through the air filter 200, the filter media 210 collects dust, antigens, and toxins. Thus, the filter toxin and antigen detector 100 would be placed on, or attached to, an air filter 200, such that air flow can pass through a portion of the detector 100, in the direction shown by air flow path arrows 130 (see FIGS. 1, 7, and 9).

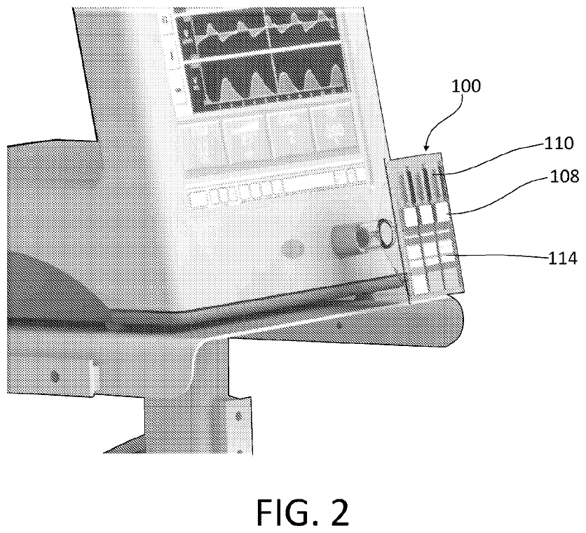

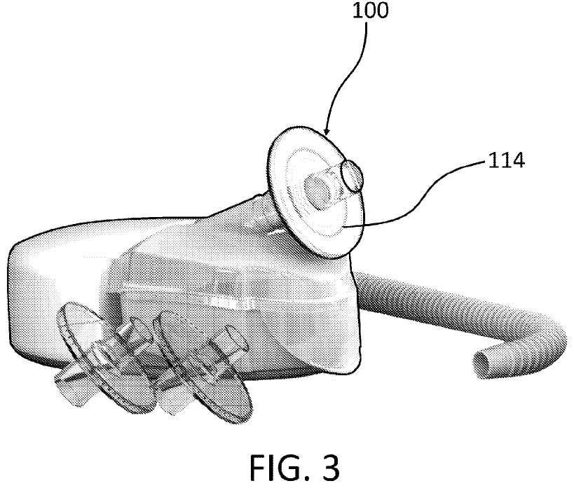

Within respiration, ventilation, or breathing devices, the toxin and antigen detector 100 may be placed on the inhalation port of the ventilator, as shown in FIG. 2, for example. For CPAP or BIPAP devices, the toxin and antigen detector 100 may take the shape of a round test cartridge 100 (cassette, or test strip) for example, and may be placed on the expiratory end of the device, as shown in FIG. 3. However, it should be understood, that the filter toxin and antigen detector 100 (or round test cartridge 100) shown and described in FIGS. 2 and 3 are exemplary only for the purposes of illustration, and may be any size and/or shape and/or may be placed anywhere (i.e., inlet, outlet, mid-device, tubing, fan, filters, etc.) within the air flow pathway of a device or system.

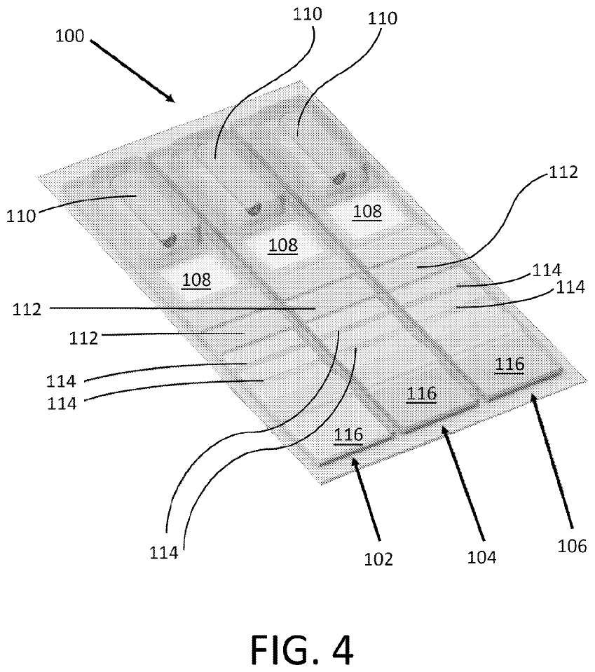

The filter toxin and antigen detectors 100 disclosed herein utilize lateral flow assay technology (known as lateral flow immunochromatographic assays/tests) having a sample pad window 132 which allows air flow therethrough, to trap particles in the air as they pass through the sample pad window 132 (for later testing/assay). FIGS. 4-7 illustrate exemplary filter toxin and antigen detectors 100 having a sample pad window 132.

Generally, a lateral flow immunochromatographic assay 130, is simple paper-based devices intended to detect the presence (or absence) of a target analyte in liquid sample (matrix) without the need for specialized and costly equipment (though many lab-based applications exist that are supported by reading equipment). One example of a well-known home medical diagnostic test using this lateral flow immunochromatographic assay would be a home pregnancy test. The technology of a lateral flow immunochromatographic assay is based on a series of capillary beds, such as pieces of porous paper, microstructure polymer, or sintered polymer, each of which has the capacity to transport fluid spontaneously.

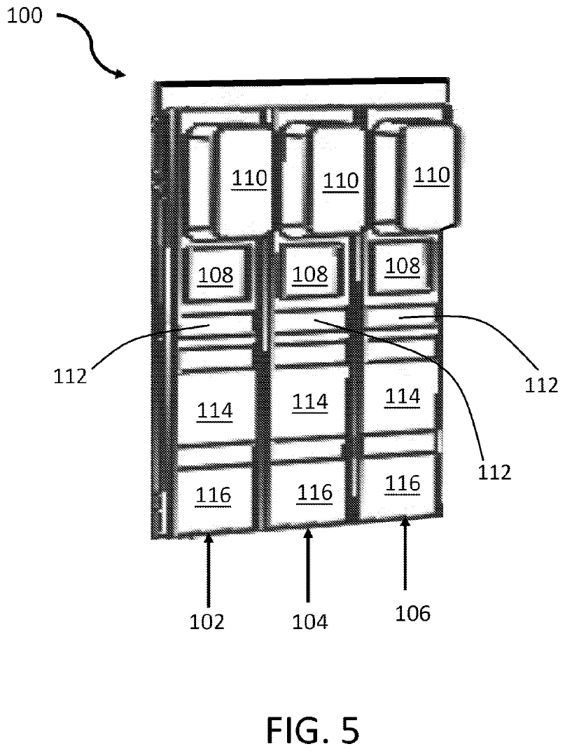

FIGS. 4 and 5 illustrate perspective views of exemplary filter toxin and antigen detectors 100. In some embodiments, each detector 100 may comprise multiple lateral flow assay/tests 102, 104, 106. For example, FIGS. 4 and 5 illustrate three separate lateral flow assay/tests 102, 104, 106 on detector 100. However, it should be understood that a detector 100 may comprise any number of lateral flow assay tests thereon. In some embodiments, each of the lateral flow assay tests 102, 104, 106 may be used to test for a different particulate, however, in other embodiments, each of the multiple lateral flow assay/tests may test for the same particulates and simply provide multiple lateral flow assay/tests on a single detector 100.

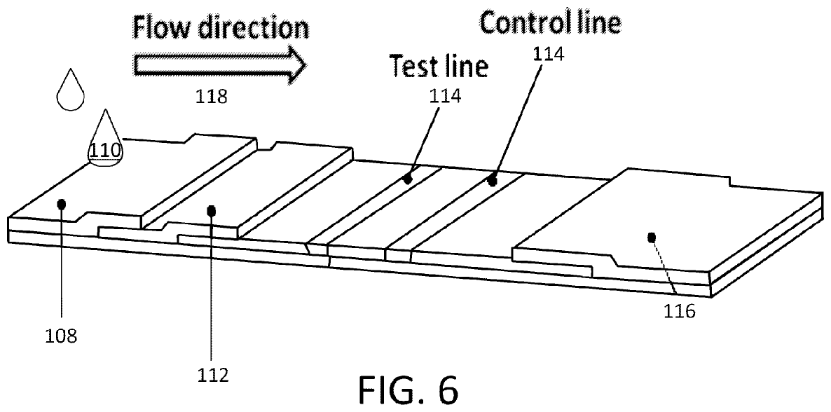

With continuing reference to the exemplary filter toxin and antigen detectors shown in FIGS. 4-7, the lateral flow immunochromatographic assay/test begins with the collection of air particulates on the sample pad window 108. Once the sample pad window 108 contains some particulates, the fluid 110, which is held within a glass ampule (also shown as 110 for clarity), is deployed (such as by crushing the glass ampule 100 with the fingers) and then migrates laterally (via spontaneous capillary action) to the conjugate pad 112, as shown in FIGS. 4-9. The fluid 110 flows laterally in the direction 118 (shown by arrows in FIG. 6). The conjugate pad 112 contains a conjugate, which is a dried form of bio-active particles in a salt-sugar matrix containing everything needed for an optimal chemical reaction between the target molecule (e.g., a harmful toxin or antigen) and its chemical partner (e.g., antibody). The conjugate (often colloidal gold) contains antibodies specific to the target toxins or antigens to be identified/detected (these targets will be predetermined by the manufacture). While the fluid 110 dissolves the salt-sugar matrix, it also dissolves the particles, and in one combined transport action, the sample and conjugate mix together while flowing through the porous matrix structure.

In this way, the analyte binds to the particles while migrating further through the third capillary bed of the lateral flow assay 102, 104, 106. The sample conjugate fluid mix then flows through an area called ‘the stripes’ 114, where a third molecule has been immobilized by the manufacturer. By the time the sample conjugate fluid mix reaches these stripes 114, analyte has been bound on the particle and the third ‘capture’ molecule binds the complex. Eventually, as more fluid mix has passed the stripes 114, particles accumulate and the stripe-area 114 changes color, providing the visible test or detector result in the form of a colored line or stripe 114 (i.e., indicating the presence or absence of harmful toxins or antigens). In some embodiments, in addition to the test result stripe 114, a control line or stripe (also shown as 114) may also be incorporated to confirm that the detector 100 is operating correctly.

After passing the stripes area 114, the fluid mix then enters the final absorbent pad 116, which simply acts as a waste receptacle to collect or absorb excess fluid. The detector 100 test results (i.e., the stripe 114 color change) will be almost immediately visible, providing a clear indication of air quality and/or device cleanliness. The stripe area 114 color change (on the detector 100) will remain visible for an extended length of time and then a user can simply properly dispose of the filter toxin and antigen detector 100 after use.

The filter toxin and antigen detectors 100 herein may be used for either qualitative or quantitative testing. While typical lateral flow assays operate on a purely qualitative basis, it is also possible to measure the intensity of the test line (i.e., the stripe area line 114) to determine the quantity of the target particulate in the sample. Using imaging processing algorithms specifically designed for a particular test type and medium, stripe line 114 intensities can be then correlated with target particulate concentration/quantity. In some embodiments, a mobile or smart phone may also be used to help in the quantification of a later flow assay/test result, such as by using the built-in camera, or light sensor, or the energy supplied by the phone's battery.

It should be noted that, in the case of respirators or ventilators, the filter toxin and antigen detector 100 may be formed in a rounded shape and thus, the stripe area 114 may be disposed in a curved or arcuate shape, instead of a more linear stripe 114 (as shown in FIG. 3). Additionally, in the case of respirators or ventilators, the later flow assay/test may not even require the fluid 110 (such as within glass ampule 11), as the moisture or humidity already present within the device may be sufficient to activate the test. In this embodiment, moisture or humidity already present within the respirator or ventilator device may activate the later flow assay/test as soon as it is inserted into the device (such as onto an inhalation port, as shown in FIG. 3).

In air flow devices having heated moisture chambers, as air from the device exits the device and is sent to the patient (such as a patient using a CPAP machine, via tubing and a mask) there is already a measurable amount of humidity within the device, which can be used as the fluid 110 necessary for activating the detector 100 test cassette, cartridge, or strip, which ultimately delivers or provides the lateral flow assay result. If a positive test result occurs (indicating the presence of a toxin or antigen), the user could then replace and/or clean or sterilize portions of the air flow device, so as not to spread or exacerbate any sort of infection or illness due to the presence of the toxin or antigen within the device.

In the case of air handling systems, a consumer could activate/start the filter and antigen detector 100 each time an air filter is exchanged, thus adding it as part of routine maintenance. In the case of respirators or other ventilation machines, a user would active/start a filter and antigen detector 100 after a patient is finished with a machine, to ensure it is clean for the next patient. In the case of respirators or other ventilation machines, the filter toxin and antigen detector 100 may be in the form of a round cassette, test cartridge, or test strip which can be activated to test cleanliness after each use/patient.

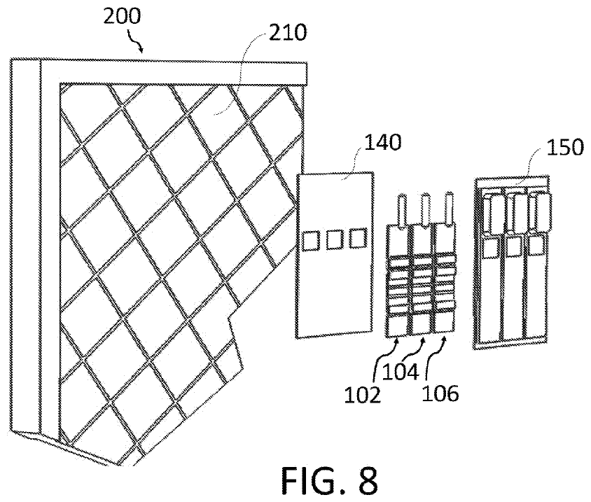

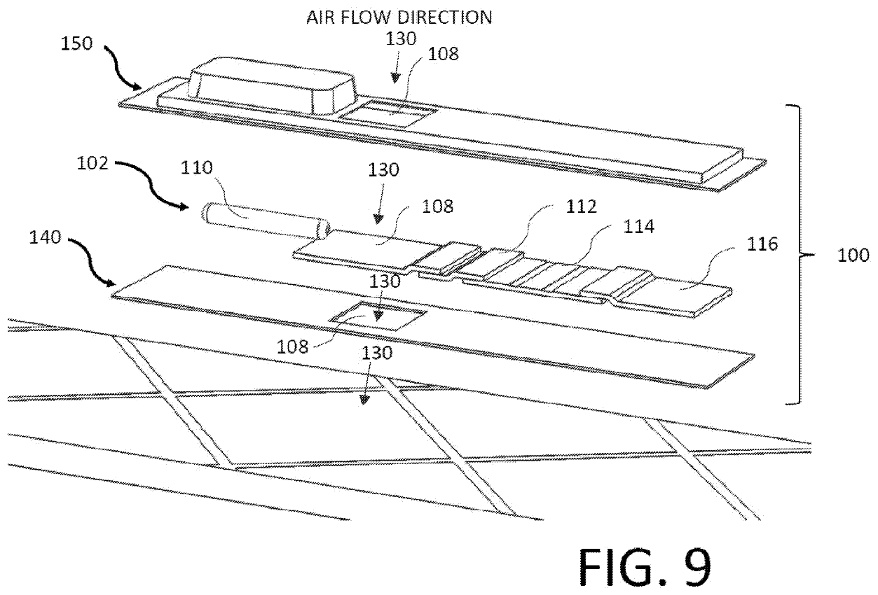

In the case of air handling systems, the filter toxin and antigen detector 100 may be attached to an air filter 200, as shown in FIG. 8, such as with an adhesive backing 140, or a hook and loop fastening system (such as Velcro), or another mechanical means affixed to the detector 100. The detector 100 itself may be designed with an integral an adhesive backing 140 and/or it may be added later by a user, such that it can be specific to each air filter 200 or airborne environment. Additionally, as shown in FIGS. 8 and 9, each lateral flow assay/test 102, 104, 106 may also have a protective overlay 150 thereon, to protect the test parts from damage, thus protecting the integrity and reliability of the lateral flow assay/tests 102, 104, 106. In some embodiments, the protective overlay 150 may be a thin piece of plastic.

The filter toxin and antigen detectors 100 disclosed herein may be used to detect particulates such as, but not limited to, antibodies, antigens, toxins, biomarkers, pathogens, viruses, bacteria, asbestos, radon, radiation, lead, pollen, pet dander, and/or other allergens. Some examples may include, but are not limited to, detection of: mold spores, Legionnaires, Legionella, norovirus, Tuberculosis, anthrax, smallpox, Ebola virus, pneumonia (ventilator associated), bacterial pathogens, staphylococcus aureur, actinobacteria species, Pseudomonas aeruginosa, Stenotrophomonas maltophilia, Klebsiella pneumoniae, Serratia marcescens, Escherichia coli (E-coli), etc.

In at least one method of operating the filter toxin and antigen detector 100 to detect the presence of harmful particulates, the method comprises positioning the detector 100 relative to a substrate (like a furnace filter 200, for example) and/or an air circulatory source (such as a fan, a furnace, an air conditioner, etc.) and activating the detector 100 by facilitating the release of a liquid 110 from the glass ampule 110 (or other liquid container) so that the liquid 110 contacts a detection element (i.e., sample pad 108 configured to collect air particulates, etc.) and then continues to contact a conjugate pad 112 etc., so that a potential chemical reaction occurs to potentially change a color of the stripe area 114 as an indicator of the presence of toxins or antigens in an airborne environment.

While various embodiments of devices and systems and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A filter toxin and antigen detector for detecting the presence or absence of target airborne particles, comprising:
at least one lateral flow immunochromatographic assay having a filter sample window therein, the filter sample window configured to collect target airborne particles flowing through an airborne environment for use in the at least one lateral flow immunochromatographic assay; and
wherein the at least one lateral flow immunochromatographic assay indicates the presence or absence of the target airborne particles.

2. (canceled)

3. (canceled)

4. The detector of claim 1, wherein the target airborne particles comprise at least one of antibodies, antigens, toxins, biomarkers, pathogens, viruses, bacteria, asbestos, lead, radiation, radon, pollen, pet dander, or allergens.

5. The detector of claim 1, wherein the filter sample window is disposed at an angle at or approximately 90 degrees relative to an airflow pathway through the airborne environment.

6. The detector of claim 1, wherein presence or absence of the target airborne particles is indicated by a color change.

7. The detector of claim 1, wherein the at least one lateral flow immunochromatographic assay is based on a series of capillary beds for transporting fluid spontaneously.

8. The detector of claim 7, wherein the at least one lateral flow immunochromatographic assay further comprises:
a fluid configured to activate the at least one lateral flow immunochromatographic assay;
a conjugate pad having antibodies specific to the airborne particles, such that a chemical reaction is initiated between the antibodies in the conjugate pad and the fluid containing the airborne particles; and
a test result stripe, wherein the chemical reaction and the fluid passing through the test result stripe cause a color change in the test result stripe, wherein the color change is indicative of the presence or absence of the target airborne particles.

9. The detector of claim 8, wherein the fluid is a liquid sample releasably stored within a glass ampule, wherein the liquid sample is deployed by crushing the glass ampule so that it may flow spontaneously.

10. (canceled)

11. (canceled)

12. (canceled)

13. (canceled)

14. The detector of claim 8, further comprising:
a control stripe adjacent the test result stripe, the control figure used to ensure the detector is operating properly.

15. The detector of claim 8, further comprising:
a thin plastic overlay to protect the integrity of the lateral flow immunochromatographic assay.

16. The detector of claim 1, further comprising an adhesive backing for attaching the detector to another surface.

17. The detector of claim 1, further comprising a hook and loop fastener for attaching the detector to another surface.

18. (canceled)

19. (canceled)

20. The detector of claim 1, wherein the at least one lateral flow immunochromatographic assay comprises multiple lateral flow immunochromatographic assays, and wherein each lateral flow immunochromatographic assay of the multiple lateral flow immunochromatographic assays tests for different target airborne particles, thus providing multiple unique tests on the detector.

21. (canceled)

22. A method of using the filter toxin and antigen detector of claim 1 to test for the target particulates within an airborne environment, comprising the steps of:
positioning the detector relative to a substrate and so that the filter sample window is within an airflow path of the airborne environment;
activating the detector by exposing it to a fluid, wherein the fluid contacts the filter sample window to initiate a chemical reaction; and
reading a visual indication on the detector to determine if the target particulates are present or absent within the airborne environment.

23. The method of claim 22, wherein the step of activating the detector is further performed by facilitating the release of the fluid from within a glass ampule, by crushing the glass ampule to release the fluid.

24. The method of claim 22, wherein the step of activating the detector further comprises exposing the detector to moisture or humidity.

25. (canceled)

26. (canceled)

27. (canceled)

28. (canceled)

29. (canceled)

30. (canceled)

31. (canceled)

32. The method of claim 22, wherein the chemical reaction is initiated when the fluid contacts the filter sample window and a conjugate within a conjugate pad of the at least one lateral flow immunochromatographic assay, wherein the conjugate pad contains antibodies specific to the target particulates within the airborne environment.

33. A filter toxin and antigen detector for detecting the presence or absence of target airborne particles, comprising:
at least one lateral flow immunochromatographic assay based on a series of capillary beds for transporting fluid spontaneously, comprising:
a filter sample window therein to collect airborne particles within an airflow pathway of an airborne environment;
a fluid configured to activate the at least one lateral flow immunochromatographic assay;
a conjugate pad having antibodies specific to the target airborne particles, such that a chemical reaction is initiated between the antibodies in the conjugate pad and the fluid containing the target airborne particles; and
a test result stripe, wherein the chemical reaction and fluids passing through the test result stripe cause a color change in the test result stripe, wherein the color change is indicative of the presence or absence of the target airborne particles.

34. The detector of claim 33, wherein the fluid is a liquid sample releasably stored within a glass ampule, wherein the liquid sample is deployed by crushing the glass ampule so that it may flow spontaneously.

35. The detector of claim 33, wherein the fluid configured to activate the at least one lateral flow immunochromatographic assay comprises moisture or humidity already present within an airborne environment.

36. A method of using a filter toxin and antigen detector to test for target particulates within an airborne environment, comprising:
positioning the detector relative to a substrate, wherein the detector comprises at least one filter sample window configured to collect particulates from the airborne environment, and wherein the filter sample window is within an airflow path of the airborne environment;
activating the detector by exposing it to a fluid, wherein the fluid contacts the filter sample window and a chemical reaction begins; and
reading a visual indication on the detector to determine if the specific particulates are present or absent within the airborne environment.
